(19) United States
(12) Patent Application Publication
(10) Pub. No.: US 2024/0181171 A1
(43) Pub. Date: Jun. 6, 2024

(54) SAFETY SYRINGE WITH SIDE COVER

(71) Applicant: BENCHA INTERNATIONAL GROUP INC., Taipei City (TW)

(72) Inventor: Lee LIN LEE, Taipei City (TW)

(21) Appl. No.: 18/525,436

(22) Filed: Nov. 30, 2023

(30) Foreign Application Priority Data
Dec. 2, 2022 (TW) 111146298

(51) Int. Cl. A61M 5/32

(52) U.S. Cl. CPC A61M 5/3216; A61M 2005/3217

(57) ABSTRACT

A safety syringe with side cover includes a needle cap, an injection needle, a needle hub, a barrel, a plunger, and a side cover, in which the needle cap is used to cover the injection needle to prevent the injection needle from being polluted by the outside and protect the user from being stuck by the injection needle before use. The side cover is engaged with the needle hub. After the operation of injecting process is completed, the side cover is turned toward the direction of the injection needle, so that the injection needle is accommodated in the accommodating recess of the side cover, thereby the user or medical staff is prevented from being injured by the exposed injection needle.

### CROSS-REFERENCE TO RELATED APPLICATIONS

This application claims the benefit of Taiwan Patent Application No. TW111146298, filed Dec. 2, 2022, which is incorporated in its entirety by reference herein.

### FIELD OF THE INVENTION

The present invention relates to a safety syringe, particularly relates to a safety syringe with side cover having low amount liquid medicine residue therein.

### BACKGROUND OF THE INVENTION

The current injection syringes generally include a needle cap, a barrel, a needle hub, an injection needle, a rubber stopper, and a plunger. In traditional technology, the front end of the syringe is provided with an injection needle. In the actual process, the safety of the injection needle after use has always been a problem issue. According to the traditional medical injection method, the needle cap is recovered to cap the injection needle again after injection process to prevent the patients and/or medical staff from being infected by accidental stab wounds. Nevertheless, the action of putting the needle cap to the injection needle may become the main cause of needle stick injuries to medical staff. In addition, if the control process of the disposal of used syringes before they are discarded is not strict enough, the injection needle may also be exposed and the cleaning staff may be injured by needle sticks, and the used syringes may also be stolen by unscrupulous people for recycle use.

### SUMMARY OF THE INVENTION

According to the disadvantages of the prior art, an object of the present invention is to provide a safety syringe with side cover, in which the side cover is attached to an outer sidewall of the needle hub. After the operation of injecting process is completed, the user or medical staff turns the side cover toward the direction of the injection needle, so the injection needle is accommodated in the accommodating groove of the side cover, and is buckled by the first fastener and the second fastener, and a pair of stoppers at the proximal end of the side cover is engaged with the pair of second ribs of the needle hub, thereby, the user or medical staff did not put the needle cap to recover the injection needle as conventional syringe. Accordingly, the safety syringe of this present invention can solve the technical problem in the prior art, when the user recovers the needle cap to the injection needle, the user and/or the medical waste disposal personnel suffer from the needle stick injury due to the needle cap is not recovered properly.

It is another object of the present invention to provide a safety syringe with side cover. The needle hub is assembled with the male luer lock connector at the distal end of the barrel by using the lugs on the outer sidewall of the needle hub and the double thread structure of the inner wall of the male luer lock connector to achieve the airtightness and the watertightness.

It is a further object of the present invention to provide a safety syringe with side cover, in which when the top end of the male luer lock connector is as close as possible to the top surface of the inner cavity of the base body of the needle hub, then the residual amount of the liquid medicine to be injected between the barrel and the needle hub tends to be lowest, which solves the technical problem of the residual liquid remaining in the needle hub in the prior art.

It is a further object of the present invention to provide a safety syringe with side cover. When the needle hub is assembled the male luer lock connector at the distal end of the barrel, the shaft seat on the outer sidewall of the needle hub is protruded out the wall of the annular wall at the distal end of the barrel. After the connecting element of the side cover is joined the shaft seat of the needle hub, the side cover turns towards the directions of the distal end and proximal end of the barrel, and the angle of the rotation range up to 180 degrees, and it can be adjusted so that the side cover will not affect the sight of the user and the injection operations. After the operation of injecting process is completed, the side cover turns toward the injection needle at the distal end of the barrel and applied an appropriate force to the surface of the side cover, the injection needle is accommodated in the accommodating groove of the side cover to cover the injection needle, so the medical staff or waste disposal personnel may protect from the needle stick injuries.

It is a further object of the present invention to provide a safety syringe with side cover. An annular wall is arranged on the plunger to tightly fit the inner wall of the hollow body of the barrel to produce the airtightness and watertightness.

It is a further object of the present invention to provide a safety syringe with side cover. When the plunger in the barrel is pushed towards the direction of the injection needle, the liquid medicine to be injected is discharged from the injection needle by the thrust of the plunger. The rod head of the plunger is inserted into the nozzle of the male luer lock connector of the barrel, the annular stopper of the rod head rests against a bottom of the nozzle of the male luer lock connector. The liquid medicine or gas to be injected may discharge from the distal end of the injection needle by the annual stopper of the plunger. When the rod head of the plunger is inserted into the nozzle of the male luer lock connector and annual stopper resets against the bottom of the nozzle of the male luer lock connector, the edge of the plunger fits tightly against the inner wall the distal end of the hollow body of the barrel, so the residual amount of the liquid medicine in the hollow body of the barrel and in the distal end of the male luer lock connector is lowest to solve the problem in the prior art, when the rubber without rod head on the top of the plunger discharges the liquid to be injected from the distal end of the injection needle, the liquid medicine remains in the channel of the male luer lock connector at the distal end of the barrel.

According to the above objects, the present invention provides a safety syringe with side cover, which includes a needle cap, a barrel, a needle hub, an injection needle, a plunger, and a side cover. The barrel having a distal end and a proximal end, and is composed of a hollow body, an annular wall at the distal end of the barrel, and a male luer lock connector surrounded by the annular wall. An inner wall of the annular wall is provided with a double thread structure, and there is a nozzle in the male luer lock connector, which communicates with the hollow body.

The needle hub having a distal end and a proximal end, and is composed of a pillar, a base seat, and a base body. The pillar is arranged on the base seat and is adjacent to the distal end of the needle hub. The base body is adjacent to the proximal end of the needle hub. There is a pillar passage in the pillar, and the outer sidewall of the pillar having a pair of first ribs and a pair of second ribs. The pair of second ribs includes a concave portion respectively. An inner cavity in the base body and an opening end of the inner cavity is facing towards the proximal end of the needle hub. The outer sidewall adjacent to a top end of the base body is provided with a shaft seat transversally protruding out the outer sidewall. The shaft seat includes an opening facing the proximal end of the needle hub, and a pair of laterally protruding lugs are provided on the outer sidewall adjacent to the bottom of the base body.

When the needle hub is combined with the male luer lock connector of the barrel, the pair of lugs on the outer sidewall of the base body of the needle hub rotate along the double thread structure on the inner wall of the annular wall and lock the needle hub on the male luer lock connector of the barrel tightly, so the shaft seat on the outer sidewall of the base body of the needle hub extends and protrudes outside the wall body of the annular wall. A gap is maintained between a bottom end of the shaft seat and the top end of the annular wall of the barrel, and this gap makes the shaft seat and the annual wall not in contact with each other. Thereby, the pillar passage in the pillar of the needle hub, the nozzle of the male luer lock connector and the hollow body of the barrel are connected to form a fluid channel.

The injection needle is arranged in a pillar passage of the pillar of the needle hub.

The plunger has a distal end and a proximal end, and is composed of a rod head, an annular stopper, and a rod body. The annular stopper is arranged between the rod head and the rod body. The plunger is placed in the barrel and is pushed from the proximal end of the barrel to the distal end of the barrel. The rod head adjacent to the distal end of the plunger is inserted into the nozzle of the male luer lock connector at the distal end of the barrel, so that a top end of the rod head may reach the top end of the male luer lock connector. The annular stopper of the rod head rests against the bottom of the nozzle toward the male luer lock connector in the hollow body of the barrel, and an edge of the annular stopper fits tightly against an inner wall of the hollow body of the barrel.

The side cover has a distal end and a proximal end, and there is a top plate at the distal end of the side cover and a bottom plate at the proximal end of the side cover. The side cover has an accommodating groove. Adjacent to the proximal end of the side cover, there are a first fastener, a second fastener, and a guide element on the opposite inner sidewall of the accommodating groove. The bottom plate adjacent to the proximal end of the side cover has a connecting element protruding outward and away from the side cover. The connecting element has a support frame and a rolling shaft. The rolling shaft is engaged with the support frame and there is a gap between the rolling shaft and the support frame, in which, the accommodating groove of the side cover faces the direction of the needle hub, and the rolling shaft of the connecting element of the side cover is fastened into the opening of the shaft seat of the needle hub, so that the side cover can turn towards the direction of the proximal end or the distal end of the needle hub through the connecting element through the shaft seat.

According to the above objects, the present invention further provides a needle hub with a side cover, which includes the needle hub and the side cover. The needle hub has a distal end and a proximal end, and is composed of a pillar, a base seat, and a base body. The pillar is arranged on the base seat and adjacent to the distal end of the needle hub, and the base body is adjacent to the proximal end of the needle hub. There are a pair of first ribs and a pair of second ribs on the outer sidewall of the pillar, and each the pair of second ribs has concave portion respectively. The base body has an inner cavity, and an opening end of the inner cavity is facing toward the proximal end of the needle hub. An outer sidewall adjacent to a top end of the base body is provided with a shaft seat laterally protruding out the outer sidewall. The shaft seat having an opening facing toward the proximal end of the needle hub, a pair of laterally protruding lugs on the outer sidewall adjacent to the bottom end of the base body.

The side cover has a distal end and a proximal end, and there is a top plate at the distal end of the side cover and a bottom plate at the proximal end of the side cover. The side cover has an accommodating groove. Adjacent to the proximal end of the side cover, there are a first fastener, a second fastener and a guide element on the opposite inner sidewall of the accommodating grove. The bottom plate adjacent to the proximal end of the side cover has a connecting element protruding outward and away from the side cover. The connecting element has a support frame and a rolling shaft. The rolling shaft is engaged with the support frame and there is a gap between the rolling shaft and the support frame. When the accommodating groove of the side cover faces the direction of the needle hub, and the rolling shaft of the connecting element of the side cover is fastened into the opening of the shaft seat of the needle hub, so that the side cover can turn towards the direction of an injection hub through the connecting element through the shaft seat.

### BRIEF DESCRIPTION OF THE DRAWINGS

FIG. 1 is a schematic diagram of showing the three-dimensional structure of the safety syringe with side cover in accordance with the present invention disclosed herein;

FIG. 2 is a schematic diagram of showing the three-dimensional structure of the needle cap in accordance with the present invention disclosed herein;

FIG. 3A is a schematic diagram of showing the three-dimensional structure of the needle hub in accordance with the present invention disclosed herein;

FIG. 3B is a side view of showing schematic diagram of three-dimensional structure of the needle hub in accordance with the present invention disclosed herein;

FIG. 3C is a top view of showing schematic diagram of a bottom view of the needle hub in accordance with the present invention disclosed herein;

FIG. 3D is a cross-sectional view of showing schematic diagram of needle hub in accordance with the present invention disclosed herein;

FIG. 4 is a schematic diagram of showing the three-dimensional structure and a cross-sectional view of the barrel in accordance with the present invention disclosed herein;

FIG. 5 is a schematic diagram of showing the three-dimensional structure of the plunger in accordance with the present invention disclosed herein;

FIG. 6A is a schematic diagram of showing the cover plate of the side cover in accordance with the present invention disclosed herein;

FIG. 6B is a schematic diagram of showing the structure of the side cover provided with the accommodating groove in accordance with the present invention disclosed herein;

FIG. 6C is a schematic diagram of the structure of the shaft connector of the side cover provided with the connecting element in accordance with the present invention disclosed herein;

FIG. 7 is a schematic diagram of showing the three-dimensional structure of the safety syringe with side cover before the operation of the injecting process in accordance with the present invention disclosed herein;

FIG. 8A is a schematic diagram of showing the side view of the side cover sleeving the injection needle after the injecting operation is completed in accordance with the present invention disclosed herein;

FIG. 8B is a schematic diagram of showing the accommodating space where the injection needle is accommodated in the side cover the back side view of the side cover covering the injection needle after the injecting operation is completed in accordance with the present invention disclosed herein; and

FIG. 8C is a schematic diagram of the front side view of the side cover sleeving the injection needle after the injecting operation in accordance with the present invention disclosed herein.

### DETAILED DESCRIPTION OF THE PREFERRED EMBODIMENT

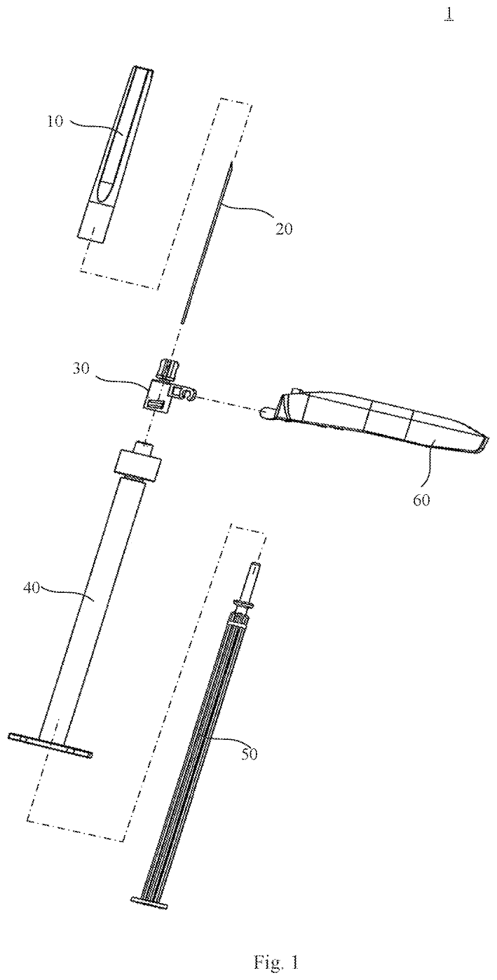

First, please refer to FIG. 1. FIG. 1 is a schematic diagram of showing the three-dimensional structure of the safety syringe with side cover. As shown in FIG. 1, the safety syringe with side cover 1 at least includes: a needle cap 10, an injection needle 20, a needle hub 30, a barrel 40, a plunger 50 and a side cover 60. The needle cap 10 is used to sleeve on the injection needle 20 to prevent the injection needle 20 from being polluted by the outside environment and protect the user from being stuck by the injection needle 20 before the safety syringe with side cover 1 is used. The injection needle 20 is arranged on the needle hub 30, and the plunger 50 is in the barrel 40 and can move back and forth in the barrel 40. The side cover 60 is engaged with the needle hub 30. After the operation of injecting process is completed, the side cover 60 is turned towards the direction of the injection needle 20 to accommodate the injection needle 20 in the side cover 60, thereby the user or others is(are) prevented from being injured by the exposed injection needle 20. In addition, it should be noted that, in the specific embodiments disclosed below, the “distal end” refers to the end away from the user, and the “proximal end” refers to the end close to the user. Each element of the safety syringe with side cover 1 disclosed in the present invention will be described in detail below.

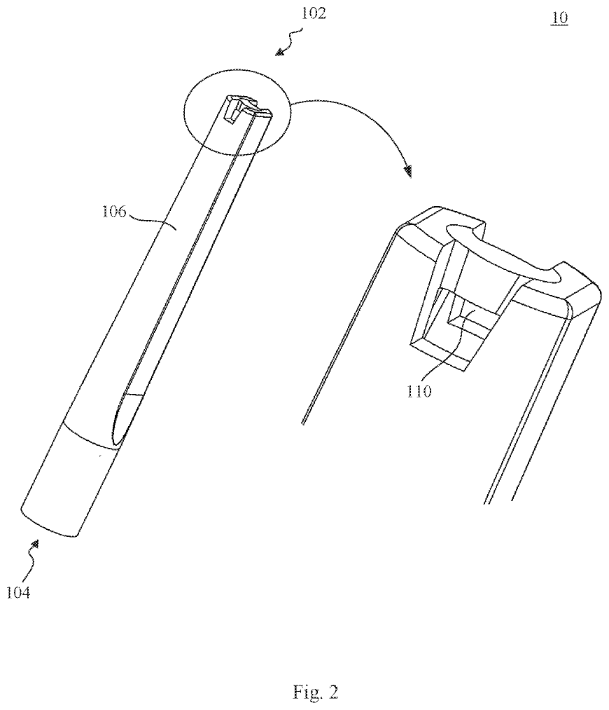

FIG. 2 is a schematic diagram of showing the three-dimensional structure of the needle cap. As shown in FIG. 2, the shape of the needle cap 10 is approximately cuboid, and it has a distal end 102 and a proximal end 104. The distal end 102 of the needle cap 10 is a closed end with at least one holes 110 and the holes 110 allow the sterilizing gas (not shown) to pass through to achieve a standard sterilization. The proximal end 104 of the needle cap 10 is an opening end, and the width of the needle cap 10 gradually increases from the distal end 102 to the proximal end 104. When the needle cap 10 is sleeved on the injection needle 20, the injection needle 20 is accommodated in the accommodating space (not shown) of the needle cap 10. In some preferred embodiments of the present invention, the surface 106 of the needle cap 10 is also treated with a roughened surface. Therefore, when the needle cap 10 in use, the user's finger (not shown) and the rough surface of the needle cap 10 to generate the friction therebetween to facilitate removal of the needle cap 10.

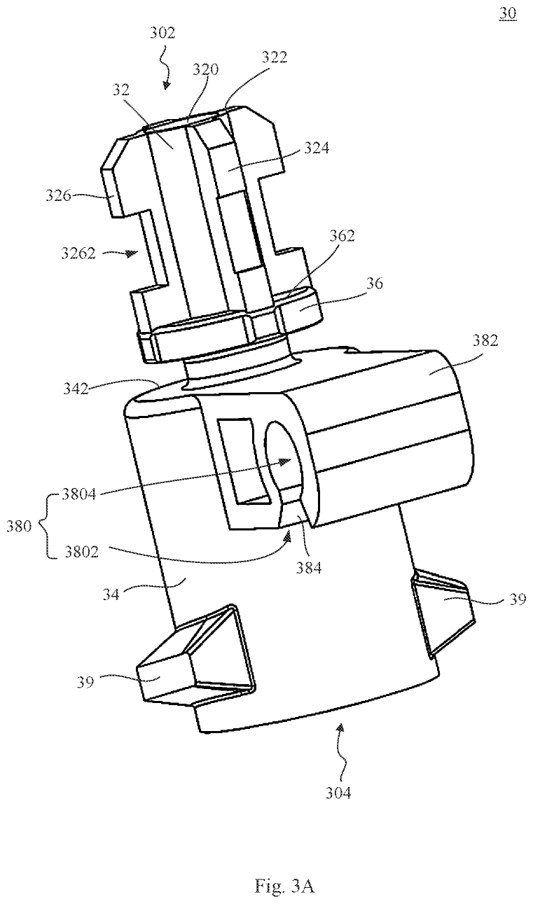

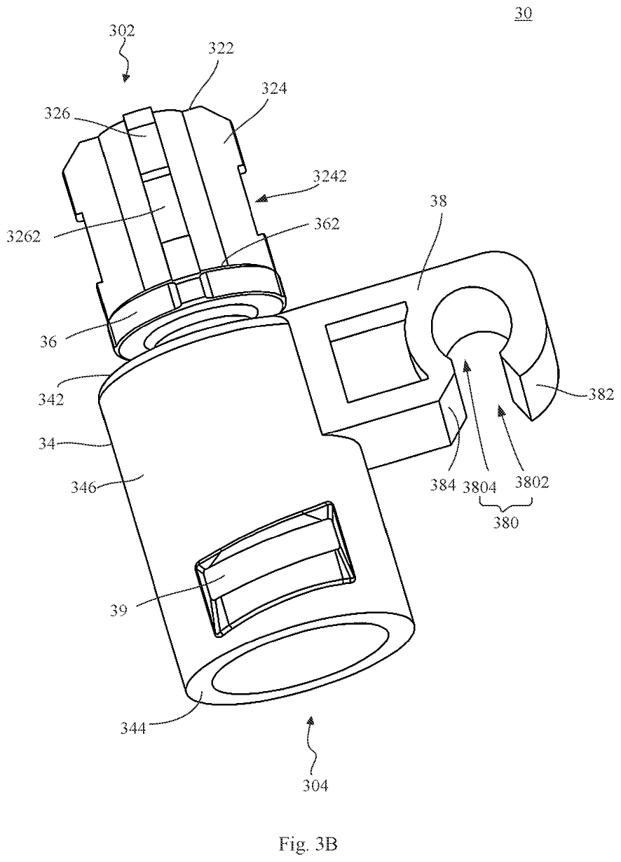

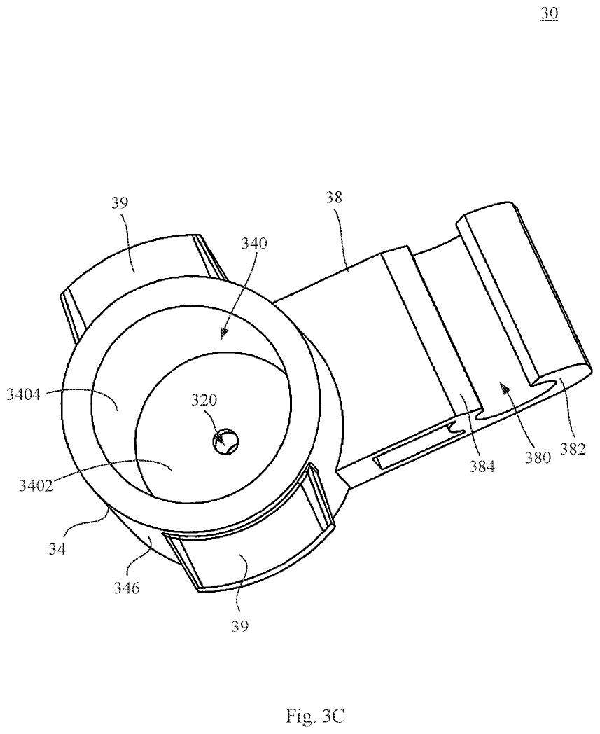

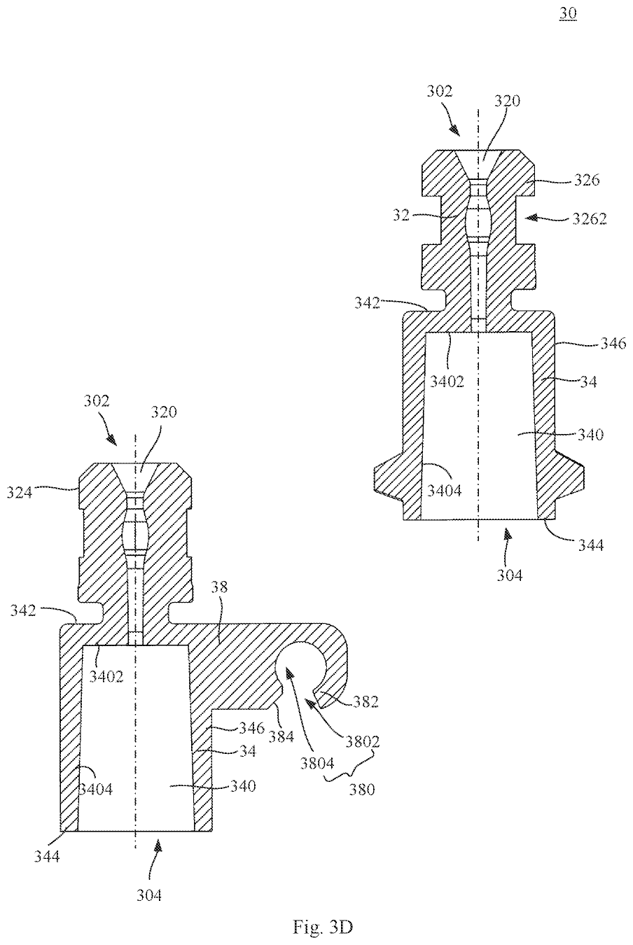

Next, please refers to FIG. 3A to FIG. 3D. FIG. 3A is a schematic diagram of showing the three-dimensional structure of the needle hub. FIG. 3B is a side view of showing schematic diagram of three-dimensional structure of the needle hub. FIG. 3C is a top view of showing schematic diagram of the needle hub. FIG. 3D is a cross-sectional view of showing schematic diagram of the needle hub. The needle hub 30 has a distal end 302 and a proximal end 304. The needle hub 30 is composed of a pillar 32, a base seat 36 and a base body 34. The pillar 32 is arranged on the base seat 36 and the bottom of the base seat 36 is connected to the base body 34. The pillar 32 is adjacent to distal end 302 of the needle hub 30 and the base body 34 is adjacent to the proximal end 304 of the needle hub 30.

There are a pair of first ribs 324 and a pair of second ribs 326 protruding out of the outer sidewall of the pillar 32. The length of the pair of first ribs 324 and the pair of second ribs 326 accounts extends downward from the top 322 of the pillar 32 to the junction of the pillar 32 and the base seat 36 (i.e., the top end 362 of the base seat 36). The pair of first ribs 324 and the pair of second ribs 326 are arranged in a cross shape, in which the outwardly protruding width of the pair of first ribs 324 and the pair of second ribs 326 is aligned with the outer diameter of the base seat 36, and the pair of second ribs 326 has a recessed portion 3262.

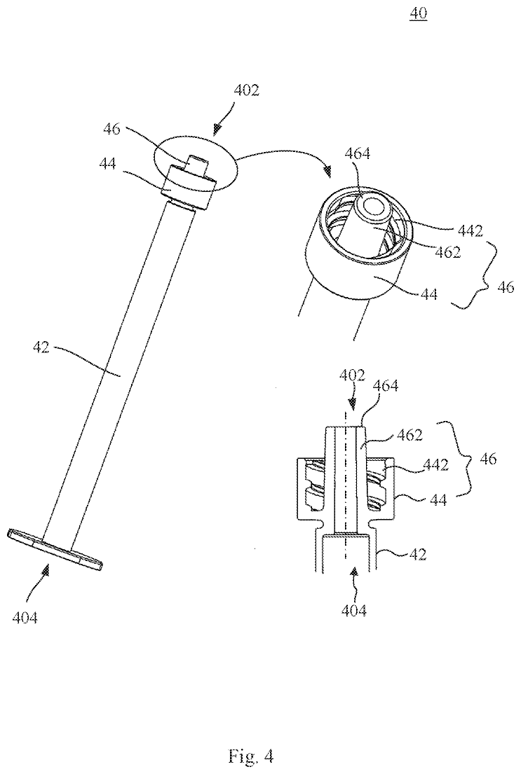

The base body 34 has an inner cavity 340, and the side wall 3404 of the inner cavity 340 has a taper, and the opening end of the inner cavity 340 faces toward the proximal end 304 of the needle hub 30. In addition, the inner cavity 340 communicates with the pillar passage 320, and the inner diameter of the pillar passage 320 of the pillar 32 is smaller than that of the inner cavity 340 of the base body 34. There is a laterally protruding shaft seat 38 on the outer sidewall 346 adjacent to the top end 342 of the base body 34, and a pair of lugs 39 adjacent to the bottom end 344 of the base body 34 (i.e., adjacent to the proximal end of the needle hub 30), in which the shaft seat 38 and the pair of lugs 39 are vertically arranged on the needle hub 30. The shaft seat 38 has an opening 380 facing the proximal end 302 of the needle hub 30. Specifically, the opening 380 of the shaft seat 38 may consider to be composed of an approximately Y-shaped notch 3802 and an approximately C-shaped notch 3804, and the opening 380 of the shaft seat 38 narrows from the approximate Y-shaped notch 3802 to the approximately C-shaped notch 3804. The pair of lugs 39 adjacent to the proximal end 304 of the needle hub 30 has an inclination angle relative to the outer sidewall 346 of the base body 34. The inclination angle is matched with that of the double-threaded structure 442 of the inner wall of the annular wall 44 of the male luer lock connector 46 of the barrel 40 (as shown in FIG. 4), and this technical content will be described in detail later.

Please refer to FIG. 4. FIG. 4 is a schematic diagram of showing the three-dimensional structure and a cross-sectional view of the barrel. As shown in FIG. 4, the barrel 40 has a distal end 402 and a proximal end 404. The barrel 40 is composed of a hollow body 42 and a male luer lock connector 46. The male luer lock connector 46 is positioned adjacent to the distal end of the barrel 40 and arranged on the hollow body 42. The male luer lock connector 46 includes an annular wall 44, an inner wall of the annular wall 44 with a symmetrical double-thread structure 442, and a nozzle 462, in which the outer wall of the nozzle 462 has a taper. The height of the male luer lock connector 46 is higher than that of the annular wall 44. This symmetrical double-threaded structure 442 has an inclination angle. The inclination angle of the pair of lugs 39 matches the inclination angle of the symmetrical double thread structure 442 at the inner wall of the annular wall 44. Therefore, when the pair of lugs 39 is engaged with the symmetrical double thread structure 442 at the inner wall of the annular wall 44, the needle hub 30 can utilize the pair of lugs 39 to rotate and lock along the symmetrical double thread structure 442 at the inner wall of the annular wall 44 to tight enough to withstand the pulling force and torsion force to meet the international standards, and the side wall 3404 of the inner cavity 340 of the base body 34 of the needle hub 30 (as shown in FIG. 3C and FIG. 3D) and the outer wall of the nozzle 462 of the male luer lock connector 46 of the barrel 40 are tightly fitted due to the same taper. Thereby, the top end 464 of the male luer lock connector 46 is as close as possible to the top surface 3402 of the inner cavity 340 of the base body 34 of the needle hub 30 to achieve watertightness and airtightness with minimum liquid residual volume between the needle hub 30 and the male luer lock connector 46.

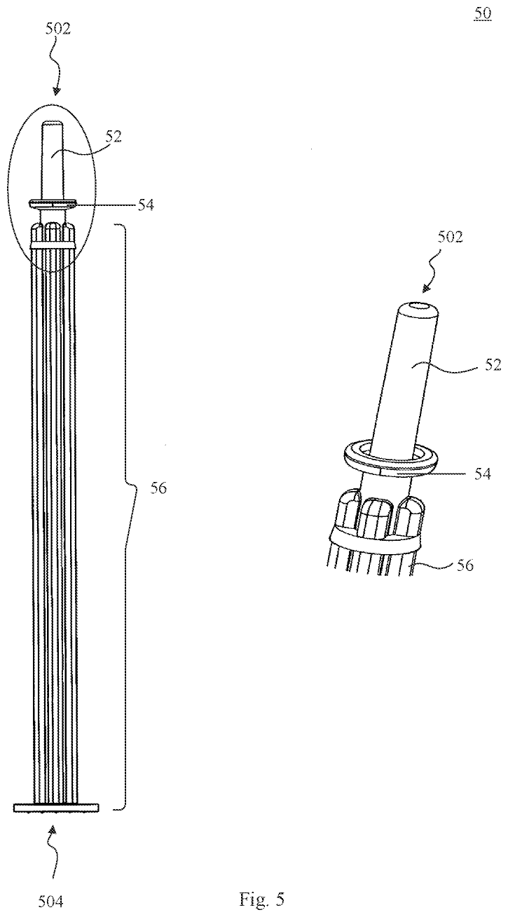

Next, please refer to FIG. 5. FIG. 5 is a schematic diagram of showing the structure of the plunger. As shown in FIG. 5, the plunger 50 is placed in the hollow body 42 of the barrel 40 as shown in FIG. 4 and push back and forth therebetween the hollow barrel 42, and the proximal 404 end and the distal end 402 of the barrel 40. The action of the plunger 50 in the barrel will describe in the following. The plunger 50 has a distal end 502 and a proximal end 504. The plunger 50 is composed of a rod head 52, an annular stopper 54 and a rod body 56. The rod head 52 of the plunger 50 is arranged on the annular stopper 54 is arranged between the rod head 52 and is adjacent to the distal end of the plunger the rod body 56. The rod body 56 connects the annular stopper 54 and is adjacent to the proximal end 504 of the plunger 50. In some embodiment of the present invention, the plunger 50 is placed in the hollow body 42 of the barrel 40 and pushed by the proximal end 404 of the barrel 40 toward the distal end 402 of the barrel 40. When the rod head 52 adjacent to the distal end 502 of the plugger 50 is inserted into the nozzle 462 of the male luer lock connector 46 of the distal end 402 of the barrel 40 and a top end of the rod head 52 reaches the same height of a top end of the male luer lock connector 46, the annular stopper 54 of the plunger 50 rests against the bottom of the nozzle 462 of the male luer lock connector 46, and an outer edge of the annular stopper 54 fits tightly against an inner wall (not shown) of the hollow body 42 of the barrel 40.

According to abovementioned, in some embodiments of this present invention, the outer diameter of the rod head 52 of the plunger 50 is smaller than the inner diameter of the nozzle 462 of the male luer lock connector 46, so the rod head 52 of the plunger 50 may insert into the nozzle 462 of the male luer lock connector 46 through the hollow body 42 of the barrel 40. The annular stopper 54 arranged around the plunger 50 abuts against the bottom of the nozzle 462 of the male luer lock connector 46, in which the outer diameter of the annular stopper 54 is larger than the inner diameter of the nozzle 462 of the male luer lock connector 46. Accordingly, while the plunger 50 in the hollow barrel 42 of the barrel 40 is pushed from the proximal end 404 of the barrel 40 toward the distal end 402 of the barrel 40, the liquid medicine to be injected (not shown) in the hollow barrel 42 of the barrel 40 is pushed by the annular stopper 54 toward the direction distal end of the barrel 40, and the liquid medicine to be injected (not shown) is pushed to flow through the fluid channel that is composed by the hollow barrel 42, the nozzle 462 of the male luer lock connector 46 and the pillar passage 320 of the pillar 32 on the needle hub 30, so the injection needle (not shown) can perform the injecting operation of the liquid medicine.

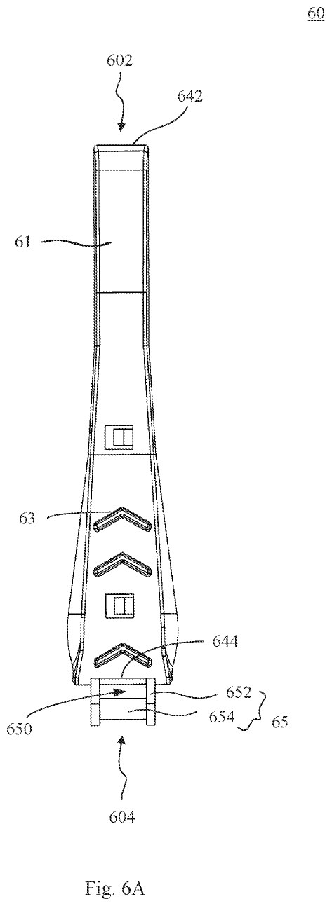

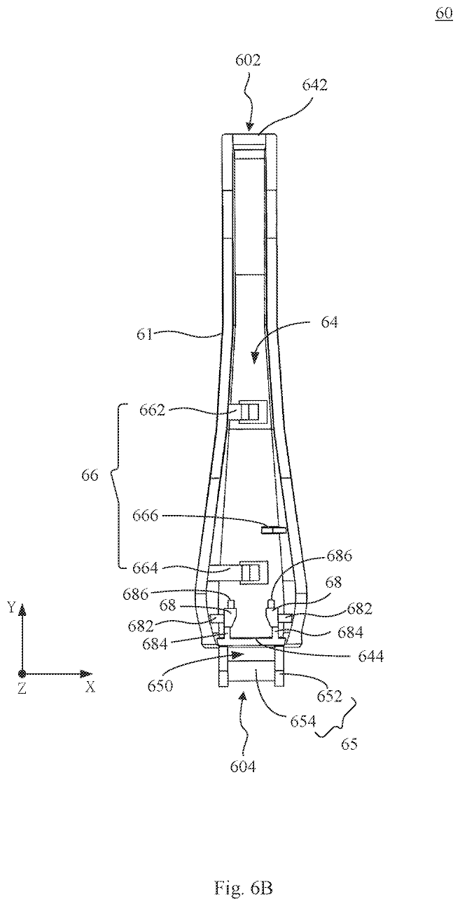

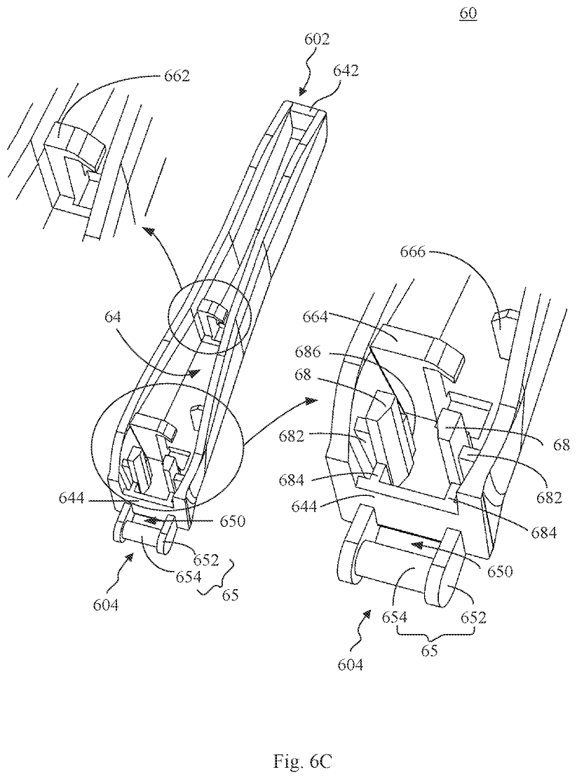

Please refer to FIG. 6A to FIG. 6C. FIG. 6A is a schematic diagram of showing the cover plate of the side cover. FIG. 6B is a schematic diagram of showing the structure of the side cover provided with the accommodating groove. FIG. 6C is a schematic diagram of showing the structure of the shaft connector of the connecting element. The side cover 60 has a distal end 602 and a proximal end 604, the distal end 602 of the side cover 60 has a top plate 642 and the proximal end 604 of the side cover 60 has a bottom plate 644. The side cover 60 has a cover plate 61 and an accommodating groove 64 is opposite to the cover plate 61. A fastener 66 is adjacent to the proximal end 604 of the side cover 60. The fastener 66 is arranged on the cover plate 61 of accommodating groove 64 and is adjacent to the two opposite inner sidewall of the accommodating groove 64, each of them respectively protruding toward to the accommodating groove 64, in which the fastener 66 is composed of a first fastener element 662, a second fastener element 664 and a guide element 666. The bottom plate 644 at the proximal end 604 of the side cover 60 has a connecting element 65 protruding outward and away from the side cover 60, in which the connecting piece 65 is composed of a support frame 652 and a rolling shaft 654. Two ends of the rolling shaft 654 are engaged with two ends of the support frame 652 respectively, so there is a gap 650 between the rolling shaft 654 and the support frame 652. There are a pair of stoppers 68 arranged on the cover plate 61 of the accommodating groove 64 and between the second fastener 664 and the connecting element 65 at the proximal end 604 of the side cover 60. The pair of stoppers 68 are respectively adjacent to the sidewall of the accommodating groove 64 of the side cover 60 are joined to the sidewall of the accommodating groove 64 of the side cover 60 by the way of the first connecting element 682 in the X-direction (wherein the X-direction is the direction parallel to the bottom plate 644), and the pair of stoppers 68 are joined to bottom plate 644 by the second connecting element 684 in Y-direction, and a stopping block 686 is on each of the pair of stoppers 68 toward the distal end of the side cover 60.

In addition, there is an anti-slip structure 63 on the surface of the cover plate 61 of the side cover 60. The anti-slip structure 63 may form by roughening the surface of the cover plate 61 to form a plurality of uneven structures thereon or the originally smooth surface of the cover plate 61 is processed into a rough surface. The purpose of this anti-slip structure 63 is to generate the friction force between the user's finger (not shown) and the surface of the cover plate 61 on the side cover 60, when the user presses the side cover 60. Accordingly, users such as medical personnel may easily press the side cover 60 to engage with the injection needle 20. In some embodiment of the present invention, the anti-slip structure 63 is a raised arrow shaped. The stopping block 686 is provided corresponding resistance when the side cover 60 turns toward the injection needle 20 to accommodate in the accommodating groove 64 of the side cover 60 to prevent the user from pressing the side cover 60 excessively to cause the side cover 60 is separated from the needle hub 30, and also to prevent the user(s) from intentionally causing the side cover 60 to detach from the needle hub 30.

Please refer to FIG. 3C and FIG. 6. When the side cover 60 assembled the needle hub 30, the accommodating groove 64 of the side cover 60 faces toward the direction of the needle hub 30, and the rolling shaft 654 of the connecting element 65 of the side cover 60 is engaged with the opening 380 of the shaft seat 38 of the needle hub 30. Since the opening 380 of the shaft seat 38 is narrowed from the proximal end 304 of the needle hub 30 towards the distal end 302 of the needle hub 30, and the top surface (not shown) of the front end 382 of the opening 380 and the top surface of the rear end 384 of the opening are inclined planes, so that the rolling shaft 654 of the connecting element 65 at the proximal end 604 of the side cover 60 may go upward along the inclined plane of the front end 382 and the rear end 384 of the opening 380 of the shaft seat 38 to engage the opening 380 of the shaft seat 38. The front end 382 of the opening 380 of the shaft seat 38 may accommodate in the gap 650 between the bottom plate 644 and the connecting element 65, so that the side cover 60 may turn toward the direction of the needle hub 30 or the direction of the barrel 40 by using the rolling shaft 654 of the connecting element 65 on the shaft seat 38, in which the maximum rotation angle is 180 degrees. In addition, it should be noted that after the side cover 60 combined with the needle hub 30, the position of the side cover 60 relative to the barrel 40 is above the side without scale (not shown), so the side cover 60 will not block the scale on the barrel 40 and will not hinder the user to perform the injection operation.

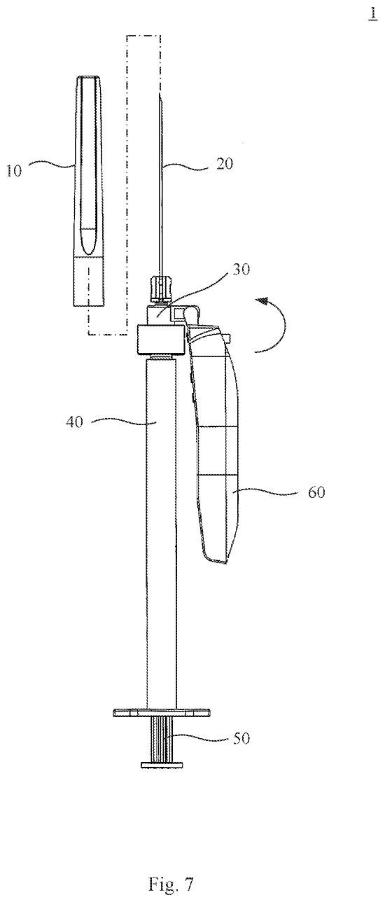

Therefore, when the above-mentioned needle hub 30 with side cover 60 is joined to the barrel 40 in FIG. 4, the nozzle 462 of the male luer lock connector 46 of the hollow barrel 40 is accommodated in the inner cavity 340 of the base body 34 of the needle hub 30. Because the taper of the outer wall of the nozzle 462 of the male luer lock connector 46 is same as the taper of the side wall 3404 of the inner cavity 340 of the base body 34 of the needle hub 30, so watertightness and airtightness can be achieved. The pair of lugs 39 (as shown in FIG. 3A) on the outer sidewall 346 of the needle hub 30 are engaged with the double thread structure 442 at the inner wall of the annular wall 44 of the male luer lock connector 46 surrounding the barrel 40, and the pair of lugs 39 rotate along the double thread structure 442, so that the needle hub 30 is set on the male luer lock connector 46 of the barrel 40. The plunger 50 in FIG. 5 is arranged in the hollow body 42 of the barrel 40. When the safety syringe with side cover 1 is not in use, a needle cap 10 is set outside the injection needle 20 to protect the injection needle 20 from external contamination and protect the user from being stabbed due to the injection needle 20 is exposed, as shown in FIG. 7.

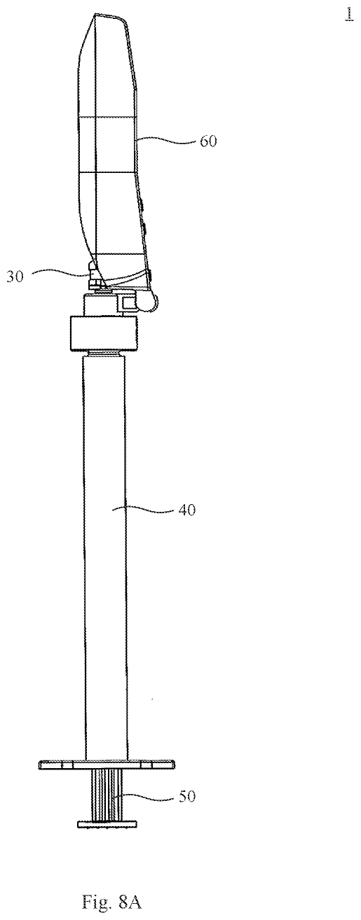

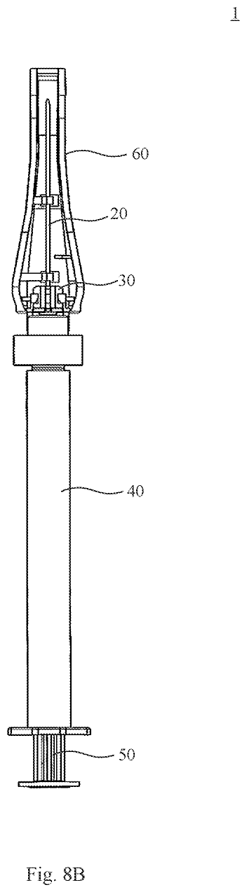

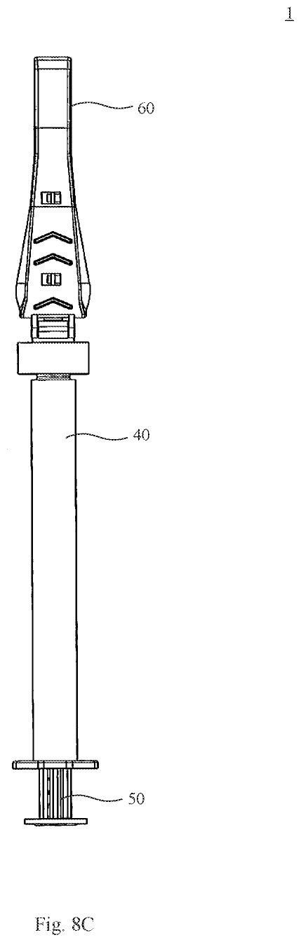

Next, please refer to FIG. 8A to FIG. 8C. FIG. 8A is a schematic diagram of showing the side view of the side cover sleeving the injection needle after the injecting operation is completed. FIG. 8B is a schematic diagram of showing the accommodating space where the injection needle is accommodated in the side cover after the injecting operation is completed. FIG. 8C is a schematic diagram of showing the front side view of the side cover sleeving the injection needle after the injecting operation is completed. As shown in FIG. 8A and FIG. 8B, after the injecting operation is completed, the side cover 60 turns towards the direction of the injection needle 20. When the side cover 60 is in contact with the injection needle 20, the surface of the cover plate 61 of the side cover 60 is pressed, so that the first fastener 662 and the second fastener 664 (as shown in FIG. 6B or FIG. 6C) in the accommodating groove 64 of the side cover 60 are in contact with the injection needle 20, and continue to apply force to the surface of the cover plate 61 of the side cover 60, so that the injection needle 20 pressed from the first fastener 662 and the second fastener 664 at the same time, and moved toward the accommodating groove 64 of the side cover 60, and the injection needle 20 is guided to the direction of the first fastener 662 and the second fastener 664 by the guide element 666, and is arranged under the first fastener 662 and the second fastener 66. The pair of the stoppers 68 in the accommodating groove in side cover 60 are laterally engaged with the recess 3262 of the pair of second ribs of the pillar 32 on the needle hub 30, in which the width of each of the pair of stoppers 68 is same as the depth of the recesses 3262 of the pair of the second ribs.

According to above-mentioned, the safety syringe with side cover 1 can utilize the side cover 60 to cover the injection needle 20 that the injecting operation is completed, so that the medical staff or the personnel handling medical waste can prevent from being injured by the exposed injection needle 20.

The above description is only preferred embodiments of the present invention, and is not intended to limit the scope of rights of the present invention; at the same time, the above description should be clear and implementable for those skilled in the relevant technical field, so other equivalent changes or modifications completed under the spirit of the invention should be included in the scope of the present application.

What is claimed is:

1. A safety syringe with side cover, comprising:
a barrel has a distal end and a proximal end, and is composed of a needle cap, a hollow body, a male luer lock connector at the distal end of the barrel, wherein the male luer lock connector with a nozzle surrounded by an annular wall is arranged on the hollow body, an inner wall of the annular wall is arranged a double thread structure, and the nozzle in the male luer lock connector, which communicates with the hollow body;
a needle hub has a distal end and a proximal end, and is composed of a pillar, a base seat, and a base body, the pillar is arranged on the base seat and adjacent to the distal end of the needle hub, the base body is adjacent to the proximal end of the needle hub, wherein an outer sidewall of the pillar has a pair of first ribs and a pair of second ribs, the pair of second ribs has a concave portion and the base body has an inner cavity, an open end of the inner cavity is facing toward the proximal end of the needle hub, an outer sidewall adjacent to a top end of the base body having a shaft seat laterally protruding out of outer sidewalls, the shaft seat having an opening facing toward the proximal end of the needle hub, a pair of lugs laterally protruding are provided on the outer sidewall adjacent to the bottom of the base body, when the needle hub is assembled with the male luer lock connector of the barrel, the pair of lugs on the outer sidewall of the base body of the needle hub rotate along the double thread structure at the inner wall of the annular wall and lock the needle hub tightly on the male luer lock connector of the barrel, so the shaft seat on the outer sidewall of the base body of the needle hub protruding out of a wall body of the annular wall, a gap is between a bottom end of the shaft seat and a top end of the annular wall to maintain the shaft seat and the annular wall not contact each other, whereby the pillar of the needle hub, the nozzle of the male luer lock connector and the hollow body of the barrel are connected to form a fluid channel;
an injection needle arranged in a pillar channel of the pillar of the needle hub;
a plunger has a distal end and a proximal end, and is composed of a rod head, an annular stopper and a rod body, wherein the annular stopper is arranged between the rod head and the rod body, the plunger is placed in the barrel and pushed from the proximal end of the barrel toward the distal end of the barrel, the rod head adjacent to the distal end of the plunger is inserted into the nozzle of the male luer lock connector at the distal end of the barrel, so a top end of the rod head reaches the same height of a top end of the male luer lock connector, the annular stopper of the rod head rests against a bottom of the nozzle toward the male luer lock connector in the hollow body of the barrel, and an edge of the annular stopper fits tightly against an inner wall of the hollow body of the barrel; and
a side cover has a distal end and a proximal end, the distal end of side cover includes a top plate and the proximal end of the side cover includes a bottom plate, the side cover having an accommodating groove, and on the opposite inner sidewall of the accommodating groove, there are respectively a first fastener, a second fastener and a guide element protruding toward the accommodating groove, the bottom plate adjacent to the proximal end of the side cover having a connecting element protruding outward and away from the side cover, the connecting element having a support frame and a rolling shaft, the rolling shaft is engaged with the support frame to form a gap between the rolling shaft and the supporting frame, wherein when the accommodating groove of the side cover faces toward the direction of the needle hub, the rolling shaft of the connecting element of the side cover is fastened into an opening of the shaft seat of the needle hub, so that the side cover rotated toward the direction of the injection needle on the needle hub and the hollow body of the barrel by the connecting element through the shaft seat.

2. The safety syringe with side cover according to claim 1, wherein when the side cover is rotated toward and contacted with the injection needle, a force is applied to a surface of the cover plate of the side cover to buckle the injection needle under the first fastener and the second fastener, so the injection needle is fully accommodated and fixed in the accommodating groove of the side cover.

3. The safety syringe with side cover according to claim 2, wherein the surface of the cover plate of the side cover includes an anti-slip structure thereon.

4. The safety syringe with side cover according to claim 3, wherein the anti-slip structure is a plurality of convex structures.

5. The safety syringe with side cover according to claim 1, wherein a pair of stoppers are arranged on the cover plate of the accommodating groove of the side cover and are adjacent to the two inner sidewalls of the accommodating groove and the pair of stoppers will be engaged with the concave portion of the second ribs of the pillar of the needle hub.

6. The safety syringe with side cover according to claim 5, wherein a width of each of the pair of stoppers matches a depth of the concave portion of each of the second ribs.

7. The needle hub with the side cover according to claim 5, further comprising a stopping block arranged on each of the pair of stoppers toward the distal end of the side cover.

8. The safety syringe with side cover according to claim 1, wherein a height of the male luer lock connector is higher than that of the wall body of the annular wall, and the height of the male luer lock connector matches that of the inner cavity of the base body of the needle hub.

9. The safety syringe with side cover to claim 1, wherein the lugs on the sidewall of the base body of the needle hub respectively having an inclination angle, and the inclination angle matches an inclination of the double thread structure of the inner wall of the male luer lock connector.

10. The safety syringe with side cover to claim 1, wherein a maximum rotation angle of the side cover between the hollow body of the barrel and the injection needle is 180 degrees.

11. A needle hub with a side cover, comprising:
the needle hub has a distal end and a proximal end, and is composed of a pillar, a base seat, and a base body, the pillar is arranged on the base seat, and is adjacent to the distal end of the needle hub, the base body is adjacent to the proximal end of the needle hub, wherein an outer sidewall of the pillar has a first pair of ribs and a second pair of ribs, the second pair of ribs has a concave portion and the base body has an inner cavity, an open end of the inner cavity is facing toward the proximal end of the needle hub, an outer sidewall adjacent to a top end of the base body is provided with a shaft seat laterally protruding out the outer sidewall, the shaft seat having an opening facing toward the proximal end of the needle hub, a pair of laterally protruding lugs on the outer sidewall adjacent to a top end of the base body; and
the side cover having a distal end and a proximal end, the distal end of the side cover includes a top end and the proximal end of the side cover includes a bottom plate, the side cover having an accommodating groove, and on the opposite inner sidewall of the accommodating groove, there are respectively a first fastener, a second fastener, and a guide element protruding toward the accommodating groove, the bottom plate adjacent to the proximal end of the side cover having a connecting element protruding outward and away from the side cover, the connecting element having a support frame and a rolling shaft, the rolling shaft is engaged with the support frame to forma a gap between the rolling shaft and the supporting frame, wherein when the accommodating groove of the side cover faces toward the direction of the needle hub, the rolling shaft of the connecting element of the side cover is fastened into the opening of the shaft seat of the needle hub, so that the side cover rotated towards the direction of an injection needle on the needle hub and the hollow body of the barrel by the connecting element through the shaft seat.

12. The needle hub with the side cover according to claim 11, wherein a maximum rotation angle of the side cover rotated toward the needle hub is 180 degrees.

13. The needle hub with the side cover according to claim 11, wherein a pair of stoppers are arranged on the cover plate of the accommodating groove of the side cover and are adjacent to the two inner sidewalls of the accommodating groove and the pair of stoppers will be engaged with the concave portion of the second ribs of the pillar of the needle hub.

14. The needle hub with the side cover according to claim 13, wherein a width of each of the pair of stoppers matches a depth of the concave portion of each of the pair of second ribs.

15. The needle hub with the side cover according to claim 13, wherein each of the pair of stoppers is arranged on the cover plate of the accommodating groove of the side cover and is connected to the sidewall of the accommodating groove of the side cover through a first connecting element and connected to the bottom plate through a second connecting element.

16. The needle hub with the side cover according to claim 13, further comprising a stopping block arranged on each of the pair of stoppers toward the distal end of the side cover.
